(19) United States
(12) Patent Application Publication
(10) Pub. No.: US 2011/0125414 A1
(43) Pub. Date: May 26, 2011

(54) METHOD FOR OPERATING A GAS SENSOR

(75) Inventors: Bernd Schumann, Rutesheim (DE); Thomas Classen, Stuttgart (DE); Berndt Cramer, Leonberg (DE)

(73) Assignee: Robert Bosch GMBH, Stuttgart (DE)

(21) Appl. No.: 12/920,985

(22) PCT Filed: Jan. 27, 2009
(86) PCT No.: PCT/EP2009/050879

§ 371 Date: Dec. 6, 2010

(30) Foreign Application Priority Data
Mar. 6, 2008 (DE) 10 2008 012 899.6

(51) Int. Cl. G01N 33/00; G06F 19/00

(52) U.S. Cl. 702/24; 73/31.05

(57) ABSTRACT

A method for operating a gas sensor is provided, wherein provision is made for determining the concentration of a gas component in a sample gas. In so doing, the gas sensor is operated in at least two different operational modes. A first operational mode (1) comprises a measurement method with at least two operations per measured value and a second operational mode (2) a faster operational mode with fewer and/or faster operations per measured value than in the first operation

### TECHNICAL FIELD

The present invention relates to a method for operating a gas sensor for determining the concentration of a gas component in a sample gas. A computer program and a computer program product, which are suited for the implementation of the method, are also the subject matter of the present invention.

Gas sensors are employed for the measuring of gas concentrations in sample gases. The determination of a gas concentration is particularly of importance with respect to exhaust gases of internal combustion engines. By making such a determination, a ratio of air to fuel can be set, which is optimal for combustion. Lambda probes are typically employed for measuring the oxygen content in the exhaust gas, in particular in motor vehicles. A lambda probe compares the residual oxygen content in the exhaust gas to the atmospheric oxygen content and transmits this value forward to a control unit usually as an analogous electrical signal.

Beside the so-called step-change probes, which operate in the range of lambda=1, i.e. at a stoichiometric ratio of air to fuel, so-called broadband lambda probes are used in diesel and Otto engines, which are also operated outside of the range of lambda=1. Broadband lambda probes can reliably acquire the residual oxygen content in the exhaust gas over a wide range. They essentially consist of a combination of a conventional concentration probe (Nernst probe) acting as a galvanic cell and a limit current or pump cell. This complex construction allows for the determination of the air/fuel ratio over a wide range; however, it also requires an increased number of electrodes, a heating element and a corresponding number of leads. A typical broadband lambda probe comprises, for example, an outer pump electrode, an inner pump electrode as well as a reference electrode and a heating element. This requires five leads, respectively wires.

For reasons of cost reduction, different approaches exist for simplifying the sensor geometry and the connection of the sensor element in the case of broad band lambda probes. This can, for example, be implemented by using a probe which has only two electrodes and four wires or even only three wires. Broadband lambda probes with a reduced number of electrodes, however, often do not allow any more for an analogous measuring principle with a continuous measuring signal as is the case with conventional lambda probes. For this reason, so-called transient measurement methods are carried out. Instead of a continuous measurement of a voltage or a current, a plurality of operations is consecutively executed in this case, with which the oxygen concentration or the concentration of another gas component can be ascertained. These operations can be measurement methods, for example: the measurement of a pump current when the pump voltage is held constant, the measurement of a voltage curve between the electrodes during current-free operation and the like. Operations can furthermore comprise, for example, pump processes, a defined oxygen quantity, for example, being pumped from one electrode to the next one. The German patent application publication DE 10 2005 006 501 A1 describes, for example, a gas sensor with two electrodes, which are activated in a clocked manner and are impressed with a potential of alternating polarity in each clock period. One to two pump processes with one to two measurement methods can, for example, be combined as a plurality of operations in a cycle when doing such transient measurement methods. An unambiguous and sufficiently accurate value for the gas concentration in the sample gas, in particular the oxygen concentration, can thereby be obtained from a corresponding calculation of the individual measured values.

With transient measurement methods, accurate values for the oxygen concentration can be obtained when there are a reduced number of electrodes on the lambda probe. The transient measurement method has, however, the disadvantage of the measured value being first obtained after the conclusion of a cycle, which comprises a plurality of operations. A corresponding period of time is therefore required so that this measurement method is relatively slow. This slow measurement method is particularly unsatisfactory when gas sensors are employed in motor vehicles. This results from the fact that rapid changes in the gas concentration, in particular rapid changes in the air coefficient lambda as a measurement for the residual oxygen content in the exhaust gas, are to be frequently acquired in said vehicles.

For this reason, the aim of the invention is to provide a method for operating a gas sensor and in particular a lambda probe, which allows for a reliable acquisition of the concentration of a gas component in the sample gas when the number of electrodes are reduced and which also meets the requirements for the acquisition of rapid changes in the concentration of the gas components in the sample gas. In this way, expanded fields of application are provided for gas sensors with a reduced number of electrodes, and the requirements for an accurate and fast measurability of gas concentrations are met. Furthermore, the complexity and the costs for the measurement of gas components in the sample gas are to be reduced with the method.

This task is solved by a method for operating a gas sensor as it is described in claim 1. Preferred embodiments of this method are described in the sub-claims.

### SUMMARY

The inventive method for operating a gas sensor serves to determine the concentration of a gas component in a sample gas. Said method is thereby characterized, in that the gas sensor is operated in at least two different operational modes. A first operational mode comprises a measurement method with at least two operations per measured value. A second operational mode comprises a faster measurement method with fewer and/or overall faster operations than in the first operational mode, for example with an operation per measured value. The first operational mode relates to a transient measurement method in the manner described above. This operational mode requires a plurality of steps, respectively operations for obtaining a measured value which reflects the concentration of the gas components in the sample gas. The operations can, for example, relate to pump processes and/or measurement methods. The plurality of steps, respectively operations, is integrated into one cycle. This cycle is run through before the measured value is obtained. An accurate measured value is obtained in this operational mode. For this purpose, a certain period of time is required, in particular the cycle duration. This is in particular a time-discrete method. This operational mode therefore operates relatively slowly.

In the second operational mode, fewer operations and/or overall faster operations than in the first operational mode are carried out to obtain a measured value. In particular one operation per measured value is carried out. This measurement method can be carried out continuously or in a time-discrete manner. A measured value can thereby be relatively quickly obtained, which is perhaps of limited validity. It is advantageous that the significance of the measured value is however sufficient in the second operational mode to determine the concentration of the gas component in the sample gas. For this reason, the inventive method for operating a gas sensor achieves the advantage that a sufficiently accurate and fast acquisition of concentrations of the gas component in the sample gas is possible by means of the combination of a relatively slow operational mode during an accurate determination of the concentration of the gas component and a second faster operational mode. This method permits the fields of application of a probe with a reduced number of electrodes, for example a lambda probe with only two electrodes, in particular in the form of a 4-wire probe or a 3-wire probe, to significantly expand. At the same time, the inventive method for operating a probe, respectively sensor, requires no or hardly any additional material or financial outlay when implementing it in a motor vehicle.

In a particularly preferred embodiment of the method according to the invention, the measured value is combined with one additional or a plurality of additional items of information in the second operational mode, which comprises fewer and/or overall faster operations than in the first operational mode, for example one operation per measured value. This process is performed for the evaluation of the measured value for determining the concentration of the gas component in the sample gas. In so doing, the significance of the measured value obtained in the second operational mode can be considerably increased. In particular the actual concentration of the gas component can thereby be reliably suggested when a measured value is, for example, ambiguous.

The additional items of information can be provided by the engine control unit in a preferred manner. The engine control unit can, for example, deliver items of information relating to the measured value to be expected. An item of information regarding the injected fuel quantity can thus, for example, suggest whether a rich or a lean air/fuel mixture with the corresponding air coefficient is to be expected. In the case of an ambiguous measured value in the second operational mode, which either indicates a rich or a lean mixture, an exact assertion can be made about the concentration of the gas component in the sample gas with the aid of this item of information from the engine control unit. In this way, a temporary double entendre or ambiguity as a result of the context of a measured value ascertained in the second operational mode can be resolved in retrospect using items of information from the engine control unit.

In an additional preferred embodiment of the method according to the invention, the additional items of information for the evaluation of the measured value in the second operational mode are provided by the control electronics of the gas sensor. Furthermore, these additional items of information can be provided by taking at least one previously determined measured value into account, for example by comparison with the previous measured value under suitable presumptions of plausibility.

In an additional preferred embodiment, a reduction of the concentration range of the gas component is used for the evaluation of the measured value, which was measured in the second operational mode. A double entendre or ambiguity of the measured value can, for example, also in this way be resolved by taking only a previously determined concentration range for, for example, the air coefficient into account during the evaluation.

Furthermore, a reduced accuracy requirement for the measured value in the second operational mode for the evaluation of the measured value can, for example, be used by, for example, allocating a greater error tolerance to this measured value. For example, a systematic error, for instance a rich shift, can furthermore be taken into account as an additional item of information for the evaluation of the measured value in the second operational mode. Said item of information can then be compensated for by a comparison with the measured values of the first operational mode.

In a particularly preferred embodiment of the method according to the invention, an operation of the first operational mode for the measurement of the measured value is used for the implementation of the second operational mode. In so doing, both operational modes can be approximately simultaneously implemented by the entire cycle of two or more operations being run through during the first operational mode before a measured value is obtained and by the individual operations of the cycle being used to achieve a measured value.

Provision can be made according to the invention for the implementation of two operational modes, as described above. It can also be preferred to implement more than two different operational modes in the method according to the invention. In this instance, provision can be made, for example, for a first operational mode, which provides very accurate measured values, with more than two operations per measured value. Provision can be made for an additional operational mode with, for example, two operations per measured value and for a third operation mode with one operation per measured value. This third operational mode allows for the measured values to be obtained very quickly but also to contain corresponding inaccuracies. The second operational mode in this embodiment allows measured values to be obtained relatively accurately and relatively quickly. Provision can be made in a corresponding manner for additional operational modes. The aforementioned description of the first and the second operational mode can accordingly be applied to the first and the additional operational modes when a plurality of operational modes is implemented. In the case of embodiments according to the invention, wherein provision is made for more than two different operational modes, gradations can be made with these additional operational modes in the accuracy, respectively reliability, and in the speed of obtaining the measured values by the employment of different operational modes. As a result of this, the advantage is achieved in that a sensor geometry, respectively a sensor arrangement, can be very flexibly adjusted and adapted to different requirements.

In an additional preferred embodiment of the method according to the invention, at least the two different operational modes of the method according to the invention can vary in the required length of time for carrying out the operations in the individual operational modes. In the first operational mode, the measurement method can, for example, comprise two operations, which are in each case relatively slow, respectively comprise a relatively long period of time. Provision can likewise be made in the second operational mode for two operations, of which at least one operation is faster, respectively requires a shorter period of time, than the operations of the first operational mode. In this embodiment, the same number of operations is carried out in both operational modes. The second operational mode is however relatively faster and also less accurate because at least one of the implemented operations of the second operational mode is faster and as the case may be less significant.

According to the invention, the process alternates between the different operational modes of the method. It is particularly preferred if the process alternates between the operational modes as a function of the concentration of the gas component in the sample gas. The first operational mode, which relatively slowly obtains measured values, can be carried out when the gas concentration does not vary much over time. When the gas concentration varies more dramatically, the second operational mode, which relatively quickly delivers measured values, can be carried out. A change in the operational mode as a function of the difference between two determined measured values can especially advantageously occur. A measurement can, for example, be taken in the first, fast and accurate operational mode as long as no significant change in the lambda value occurs, respectively is detected. This delivers an accurate as possible picture of the composition of the exhaust gas, respectively the concentration of the gas component in the sample gas. If a relatively significant difference, respectively change, is determined when comparing the last two measured values or also the partial measured values within a cycle, a step change can be made into the second operational mode, which relatively quickly provides measured values. After the alternation into the second operational mode, a possibly imprecise but fast signal is generated in comparison to the previous value from the first operational mode. If the change in the signal, respectively change in the measured value, per unit of time is again smaller, the process can alternate into the first, relatively slow but accurate operational mode.

The alternation in the operational modes can, for example, result from external signals, respectively specifications, for example with the aid of the engine control unit or by means of the control electronics of a lambda probe. The engine control unit can, for example, give the command to alternate between the first and the second operational mode via the CAN bus. Accurate lambda values can, for example, be acquired within a rich phase by measuring in the first operational mode. As soon as a step change is again made back into a lean phase, the engine control unit, in particular the lambda control electronics, gives the command to change to the second operational mode. In this way, the change can be followed in realtime. For that reason, the advantage of an open-loop control by the engine control unit is that a change in lambda does not have to be first detected with a great deal of effort but that such a change can be selectively scanned. A change between the operational modes can therefore particularly advantageously occur by an external triggering, in particular via the engine control unit. As an alternative to or in addition to said triggering, the alternation between the operational modes can result as a reaction to an analysis of the measured values. In so doing, the plausibility or the magnitude of the last change in the measured values or the variation in the measured signals can be taken into account.

In a particularly preferred embodiment of the method according to the invention, the gas sensor is a lambda probe, which is particularly provided to measure the residual oxygen content in the exhaust gas of an internal combustion engine, respectively of a motor vehicle. This lambda probe preferably relates to a lambda probe with a reduced number of electrodes, in particular a lambda probe with two electrodes. The method according to the invention can advantageously be employed with broadband lambda probes. Moreover, said method can also be employed with so-called step change probes, respectively two-point probes. In addition the method according to the invention is also applicable to other types of sensors. These sensors include, for example, nitrogen oxide analyzers or carbon monoxide sensors, which can be employed for determining the concentration of gas components in a sample gas.

The invention furthermore comprises a computer program, which executes all of the steps of the method according to the invention, if said program runs on a computer or in a control unit. Finally the invention comprises a computer program product with a program code, which is stored on a machine-readable carrier, for carrying out the described method if the program is executed on a computer or in a control unit. The inventive computer programs, respectively computer program products, can especially be advantageously employed in embodiments of motor vehicles. They are especially effective in reliably and quickly determining the concentration of gas components in the exhaust gas when sensors with a reduced number of electrodes are used.

Additional characteristics and advantages of the invention emanate from the following description of the drawings in combination with the examples of embodiment. In this regard, the individual characteristics can in each case be implemented alone or in combination with each other.

### BRIEF DESCRIPTION OF THE DRAWINGS

The following are shown in the drawings:

FIG. 1 a schematic depiction of an embodiment of the method according to the invention;

FIG. 2 a schematic progression of the oxygen content with two time-discrete operational modes according to a preferred embodiment of the method according to the invention;

FIG. 3 a schematic progression of the oxygen content with one time-discrete and one ambiguous, continuous operational mode according to a preferred embodiment of the method according to the invention.

### DETAILED DESCRIPTION

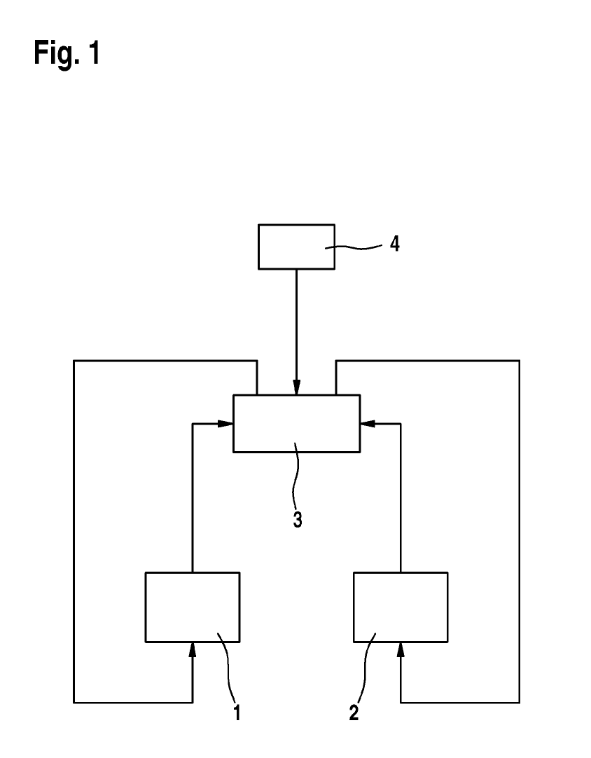

FIG. 1 schematically shows an embodiment of the method according to the invention. In so doing, a first operational mode 1 and a second operational mode 2 are depicted. The operational mode 1 comprises a measurement method with at least two operations per measured value for ascertaining the gas concentration of a gas component in a sample gas. The second operational mode 2 comprises a measurement method with fewer and/or overall faster operations than in the first operational mode, for example with one operation per measured value, for ascertaining the concentration of the gas component in the sample gas. The measurement method in the first operational mode 1 is a relatively slow measurement method, which allows for the concentration of the gas component to be accurately ascertained. The measurement method in the second operational mode 2 is a relatively fast measurement method, which likewise serves to ascertain the concentration of the gas component, which is however potentially of limited validity. The process alternates between the two operational modes so that a method for operating a gas sensor is provided as a result. Said method also allows for a sufficiently accurate and sufficiently fast determination of the concentration of the gas component in the sample gas when the configuration of the sensor is reduced in complexity, for example a reduced number of electrodes. The alternation between the operational modes 1 and 2 occurs by means of a selector element 3, for example a switch. This selector element 3 is controlled by a specification 4. This specification 4 relates, for example, to a signal from a control unit, in particular an engine control unit. Said specification 4 can furthermore relate to other external or internal signals, for example signals from the control electronics of the sensor. In addition this specification 4 can relate to measured values, which reflect the variation in the concentration of the gas component in the sample gas. A difference in two determined measured values, in particular measured values which are consecutively determined, can, for example, be used in this instance to induce a signal for an alternation between modes via the selector element 3. The first operational mode 1 can, for example, be activated in this manner when the gas concentration does not very much in the sample gas. When the gas concentration varies more dramatically, in particular when the difference between the two determined measured values is larger, the second operational mode 2 can be activated via the selector element 3. The measured values acquired in the different operational modes 1 and 2, in particular the concentrations of gas components ascertained in the process, can affect the selector element 3 as additional items of information, respectively signals.

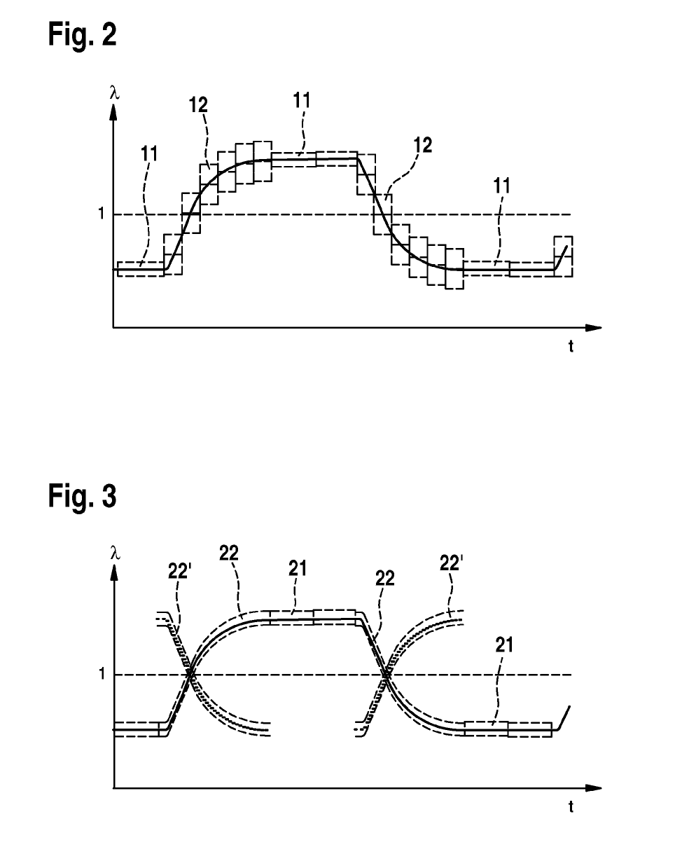

FIG. 2 schematically shows the temporal course of the air coefficient lambda, which alternates around 1. The oxygen content is measured by means of a lambda probe. According to the inventive method, lambda is measured as a measurement for the oxygen concentration in a sample gas in at least two different operational modes. In this example, both operational modes relate to a time-discrete measurement method (solid lines) with a given error range (dashed lines). This error range reflects the accuracy of the measurement of the respective operational mode. In the first operational mode 11, the error range is relatively small when the measurement interval is relatively long. In the case of the operational mode 12, the error range is relatively large when the measurement duration is relatively short. Provision is made according to the invention for the process to alternate between the two operational modes. The slower but more accurate operational mode 11 is used for ranges with slight variance of the air coefficient lambda. For the ranges with a greater variance of the air coefficient lambda, the faster but more error-prone operational mode 12 is employed.

FIG. 3 shows in a comparable manner the temporal course of the air coefficient lambda as a characteristic variable for the oxygen content in a sample gas, provision being made according to the inventive method for two different operational modes. The time-discrete first operational mode 21 delivers a relatively accurate measured value when the measurement duration is relatively long. In the second operational mode 22, the air coefficient lambda is continuously measured. This continuous measurement however yields ambiguous measured values, which are depicted as two possible allocations 22, 22′ with their respective error ranges (dashed lines). The selection of the correct allocation 22 and with it the evaluation of the measured values in the second operational mode takes place with the aid of the previously measured, time-discrete measured values, which were obtained in the relatively accurate first operational mode 21. With the aid of these items of information from the measured values of the first operational mode 21, the ambiguous measured values 22, 22′ obtained in the second operational mode can be unambiguously resolved and can be allocated to the actual oxygen concentrations.

A preferred example of embodiment for the method according to the invention uses a type of sensor, which as a broadband lambda probe for determining the residual oxygen content in the exhaust gas of an internal combustion engine has only two electrodes. This type of sensor is characterized by an approximately linear characteristic curve in the lean exhaust gas and an additional approximately linear characteristic curve in the rich exhaust gas. An unambiguous allocation of a pump current measured value is not possible with said sensor because each pump current value can be allocated to a lambda value in the lean operation as well as to a lambda value in the rich operation (V-characteristic curve). An unambiguous value for lambda can be derived from it by means of a periodic reversion of polarity of the pump voltage. This requires a specific time period T, which comprises a first phase for measurement with positive polarity, a second phase for recharging the electrodes by applying suitable negative voltages, a third phase for measurement with negative polarity and a fourth phase for recharging the electrodes by applying a suitable positive voltage. An unambiguous measured value is obtained after completing these different operations in a cycle. This operational mode, which is relatively slow, is designated as the first operational mode according to the invention. Each individual measurement of each polarity produces a measured value for lambda which is however ambiguous. If the item of information is added whether lambda is greater than or less than 1, the actual lambda value can then be unambiguously suggested with these measured values. Therefore in the second operational mode according to the invention, a measured value in only one polarization is measured, and the actual lambda value is suggested from this measured value. This remains possible as long as no lambda-1-passage takes place. Hence in the second operational mode, measurement is taken only in one polarization, preferably in the polarization which prevails at the current moment. As long as no lambda-1-passage takes place, a continuous measurement with unambiguous evaluation of the measured values is thereby possible. If a lambda-1-passage takes place, this can be detected on the basis of the pump current first dropping and then increasing again as long as the change in lambda is constant. In a normal gasoline operation, one or a plurality of accurate measurements can occur in the rich state, respectively lean state, in the second operational mode. The sensor in the second operational mode then tracks the change in lambda, if need be with detection of the lambda-1-passage. The alternation back to the first, accurate operational mode takes place, for example, via a command of the engine control unit, which anticipates and/or detects a new static lambda value. The alternation can also take place if the pump current change over time has become small enough or if the control electronics of the lambda probe assigns the measured lambda a reliability value that is too small on the basis of the recorded history.

1. Method for operating a gas sensor for determining the concentration of a gas component in a sample gas, thereby characterized, in that the gas sensor is operated in at least two different operational modes. A first operational mode (1) comprises a measurement method with at least two operations per measured value and a second operational mode (2) a faster measurement method with fewer and/or overall faster operations per measured value than in the first operational mode.

2. Method according to claim 1, thereby characterized, in that the measured value is combined with additional items of information in the second operational mode for an evaluation of the measured value for determining the concentration of the gas component in the sample gas.

3. Method according to claim 2, thereby characterized, in that the additional items of information are provided by the engine control unit.

4. Method according to claim 2 or claim 3, thereby characterized, in that the additional items of information are provided by the control electronics of the gas sensor.

5. Method according to one of the claims 2 to 4, thereby characterized, in that the additional items of information are provided while taking at least one previously determined measured value into account.

6. Method according to one of the claims 2 to 5, thereby characterized, in that the additional items of information are a limitation of the concentration range of the gas component.

7. Method according to one of the claims 2 to 6, thereby characterized, in that an operation of the first operational mode is used for the measurement of the measured value for the second operational mode.

8. Method according to one of the preceding claims, thereby characterized, in that the process alternates between the operational modes as a function of the concentration of the gas component in the sample gas.

9. Method according to one of the preceding claims, thereby characterized, in that the first operational mode is implemented when the gas concentration in the sample gas does not vary much in the temporal course and/or the second operational mode is implemented when the gas concentration in the sample gas varies more dramatically.

10. Method according to one of the preceding claims, thereby characterized, in that the operational mode alternates as a function of the difference between two determined measured values.

11. Method according to one of the preceding claims, thereby characterized, in that an alternation of the operational mode is externally controlled, in particular by an engine control unit.

12. Method according to one of the preceding claims, thereby characterized, in that the gas sensor is a lambda probe, in particular a lambda probe with two electrodes.

13. Method according to one of the preceding claims, thereby characterized, in that the gas sensor is a nitrogen oxide analyzer.

14. Computer program, which executes all of the steps of a method according to one of the claims 1 to 13, if it runs on a computer or in a control unit.

15. Computer program product with program code, which is stored on a machine-readable carrier, for carrying out a method according to one of the claims 1 to 13 if the program is executed on a computer or in a control unit.
